(19) United States
(12) Patent Application Publication
(10) Pub. No.: US 2009/0157155 A1
(43) Pub. Date: Jun. 18, 2009

(54) GRAPHICAL DISPLAY OF ENVIRONMENTAL MEASUREMENTS FOR IMPLANTABLE THERAPIES

(75) Inventor: Kerry Bradley, Glendale, CA (US)

Correspondence Address: Vista IP Law Group LLP, 2040 MAIN STREET, 9TH FLOOR, IRVINE, CA, 92614, US

(73) Assignee: ADVANCED BIONICS CORPORATION, Sylmar, CA (US)

(21) Appl. No.: 11/959,355

(22) Filed: Dec. 18, 2007

(51) Int. Cl. A61N 1/05

(52) U.S. Cl. 607/116

(57) ABSTRACT

A method and system of providing therapy to a patient implanted with an array of electrodes is provided. The electrodes are configured for respectively providing electrical stimulation to tissue of the patient. The method comprises measuring physiological parameter information indicative of the coupling efficiencies between the respective electrodes of the array and the tissue, computing numerical values from the measured physiological parameter information, generating a chart representative of the computed numerical values, and displaying the chart to a user.

### FIELD OF THE INVENTION

The present invention relates to tissue stimulation systems, and more particularly, to a system and method for measuring environmental parameters surrounding the electrodes of such tissue stimulation systems.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems such as the Freehand system by NeuroControl (Cleveland, Ohio) have been applied to restore some functionality to paralyzed extremities in spinal cord injury patients.

Each of these implantable neurostimulation systems typically includes an electrode lead implanted at the desired stimulation site and an implantable pulse generator (IPG) implanted remotely from the stimulation site, but coupled either directly to the electrode lead or indirectly to the electrode lead via a lead extension. Thus, electrical pulses can be delivered from the neurostimulator to the stimulation electrode(s) to stimulate or activate a volume of tissue in accordance with a set of stimulation parameters and provide the desired efficacious therapy to the patient. A typical stimulation parameter set may include the electrodes that are sourcing (anodes) or returning (cathodes) the stimulation current at any given time, as well as the amplitude, duration, and rate of the stimulation pulses.

The neurostimulation system may further comprise a handheld remote control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon.

When a neurostimulation system is implanted within a patient, a fitting procedure is typically performed to ensure that the stimulation leads are properly implanted in effective locations of the patient, as well as to select one or more effective sets of stimulation parameters for the patient. Follow-up programming sessions may also be performed to reprogram the IPG, e.g., if the stimulation leads migrate from the original position.

In certain scenarios, the environment surrounding electrodes in neuromodulation therapies may be characterized using a variety of measurements, e.g., impedance, field potential, activation thresholds (perception, therapeutic, side-effect, maximum comfortable, . . . ), pressure, translucence, reflectance, pH, etc. Characterization of the environment surrounding the electrodes may be used, e.g., to ascertain whether the electrode lead is optimally, or otherwise properly, located within the patient, or may be used to program the IPG to provide a more effective therapy. For example, the Precision® SCS System, marketed by Boston Scientific Corporation, measures the impedance between each of the stimulation lead electrodes and the case of the IPG, thereby providing an indication of whether the respective electrodes are efficiently coupled to the tissue.

As illustrated in FIG. 1, the impedance values 2 are displayed to the clinician in numerical fashion (i.e., a list of 16 numerical impedance values are displayed for 16 respective electrodes 4 of an array 6). While the display illustrated in FIG. 1 provides the physician or clinician the information necessary to determine the coupling efficiency between the respective electrodes 4 and the tissue, the interpretation of the list of numerical impedance values 2 by the physician or clinician is often not straightforward or efficient in a rushed clinical or operating room environment. This may be detrimental to the therapy provided by the neuromodulation system, since the information may be ignored due to the difficulty, and thus increased time, of interpreting the impedance values.

There, thus, remains a need for an improved method and system for more efficiently displaying measurements indicating the coupling between stimulation leads and tissue to a user.

### SUMMARY OF THE INVENTION

In accordance with a first aspect of the present inventions, a method of providing therapy to a patient implanted with an array of electrodes is provided. The implanted electrode array is configured for respectively providing electrical stimulation to tissue (e.g., spinal cord tissue) of the patient. The method comprises measuring physiological parameter information indicative of the coupling efficiencies between the respective electrodes of the array and the tissue, and computing numerical values (e.g., electrical impedance values, field potential values, and evoked action potential values) from the measured physiological parameter information.

The method further comprises generating a chart (e.g., a line chart or a bar chart) representative of the computed numerical values, and displaying the chart to a user. In one method, the physiological parameter information is measured using implanted control circuitry, and the chart is displayed to the user using an external device. In this case, the method may comprise transmitting the measured physiological parameter information from the implanted control circuitry to the external device, wherein the numerical values are computed by the external device. An optional method comprises programming the implanted control circuitry with a set of stimulation parameters.

In accordance with a second aspect of the present inventions, an external device for a neurostimulation system is provided. The neurostimulation system comprises telemetry circuitry configured for receiving data from an implantable device connected to an array of electrodes. The received data is derived from physiological parameter data measured by the implantable device, and is indicative of the coupling efficiencies between respective electrodes of the array and tissue. The neurostimulation system further comprises processing circuitry configured for generating a chart (e.g., a line chart or a bar chart) representative of numerical values (e.g., electrical impedance values, field potential values, and evoked action potential values) derived from the data, and a display configured for displaying the chart to a user.

In one embodiment, the received data is the measured physiological parameter data, and the processing circuitry is configured for computing the numerical values from the measured physiological parameter data. In another embodiment, the received data comprise the numerical values. In still another embodiment, the processing circuitry is configured for programming the implantable device with a set of stimulation parameters.

In accordance with a third aspect of the present invention, another method of providing therapy to a patient implanted with an array of transducers is provided. The transducers are configured for respectively providing stimulation to tissue (e.g., spinal cord tissue) of the patient. The transducers may be electrodes, but can also take the form of other transducers. The method comprises measuring physiological parameter information indicative of the efficacy of the stimulation provided to the tissue, and computing numerical values (e.g., electrical parameter values) from the measured physiological parameter information. The method further comprises generating a chart (e.g., a line chart or a bar chart) representative of the computed numerical values, and displaying the chart to a user. The method further comprises modifying the stimulation provided by the transducers based on the displayed chart. For example, the transducers may be initially programmed based on the displayed chart, or a remedial action (such as, e.g., physically moving the transducers or reprogramming the transducers) may be performed.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a display of impedance values for the electrodes of a prior art spinal cord stimulation system;

FIG. 2 is plan view of one embodiment of a spinal cord stimulation (SCS) system arranged in accordance with the present inventions;

FIG. 3 is a plan view of the SCS system of FIG. 2 in use with a patient;

FIG. 4 is a profile view of an implantable pulse generator (IPG) used in the SCS system of FIG. 2;

FIG. 5 is a block diagram of the internal components of the IPG of FIG. 4;

FIG. 6 is a plan view of a remote control that can be used in the SCS system of FIG. 2;

FIG. 7 is a block diagram of the internal componentry of the remote control of FIG. 6;

FIG. 8 is a block diagram of the components of a clinician's programmer that can be used in the SCS system of FIG. 2;

FIG. 9 is a screen display generated by the clinician's programmer of FIG. 8; and

FIG. 10 is another screen display generated by the clinician's programmer of FIG. 8.

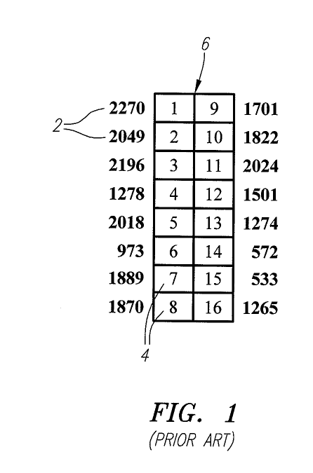

### DETAILED DESCRIPTION OF THE EMBODIMENTS

The description that follows relates to a spinal cord stimulation (SCS) system. However, it is to be understood that the while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

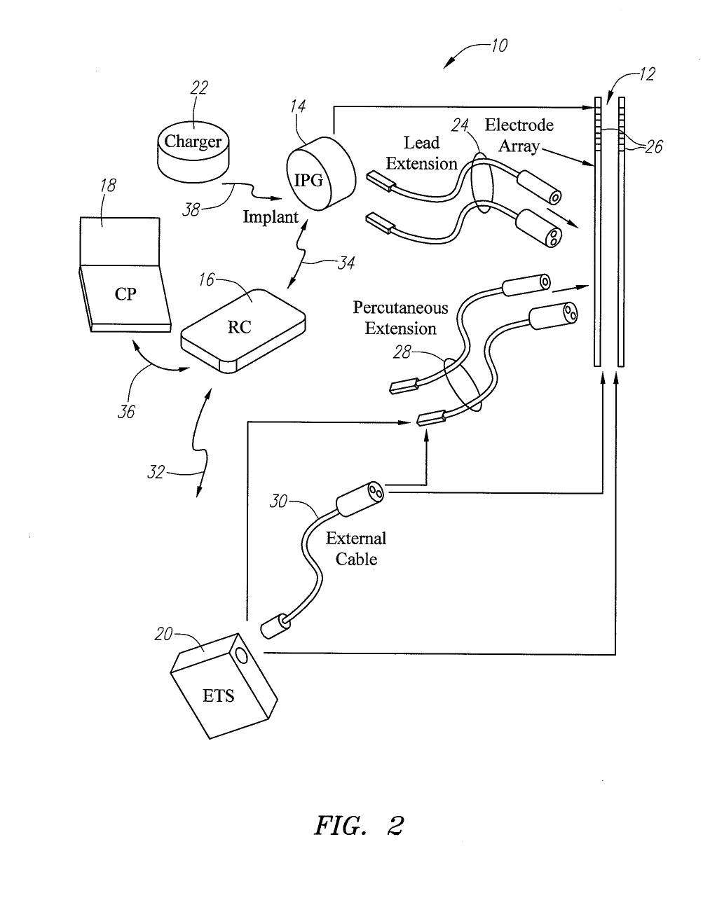

Turning first to FIG. 2, an exemplary SCS system 10 generally includes one or more (in this case, two) implantable stimulation leads 12, an implantable pulse generator (IPG) 14, an external remote controller RC 16, a clinician's programmer (CP) 18, an External Trial Stimulator (ETS) 20, and an external charger 22.

The IPG 14 is physically connected via one or more percutaneous lead extensions 24 to the stimulation leads 12, which carry a plurality of electrodes 26 arranged in an array. In the illustrated embodiment, the stimulation leads 12 are percutaneous leads, and to this end, the electrodes 26 are arranged in-line along the stimulation leads 12. In alternative embodiments, the electrodes 26 may be arranged in a two-dimensional pattern on a single paddle lead. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the stimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the stimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bidirectional RF communications link 32. Once the IPG 14 and stimulation leads 12 are implanted, the RC 16 may be used to telemetrically control the IPG 14 via a bidirectional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

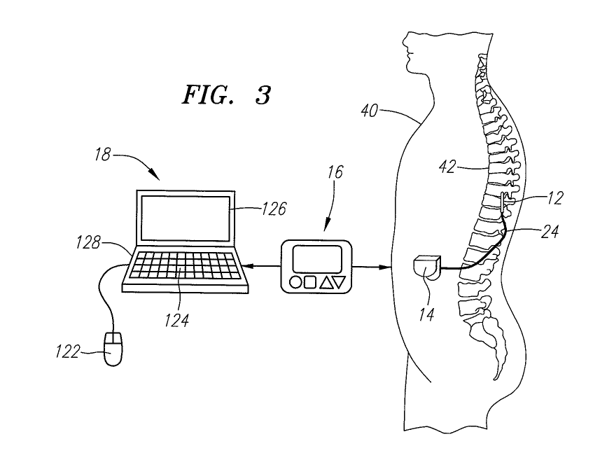

As shown in FIG. 3, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extension 24 facilitates locating the IPG 14 away from the exit point of the electrode leads 12. As there shown, the CP 18 communicates with the IPG 14 via the RC 16.

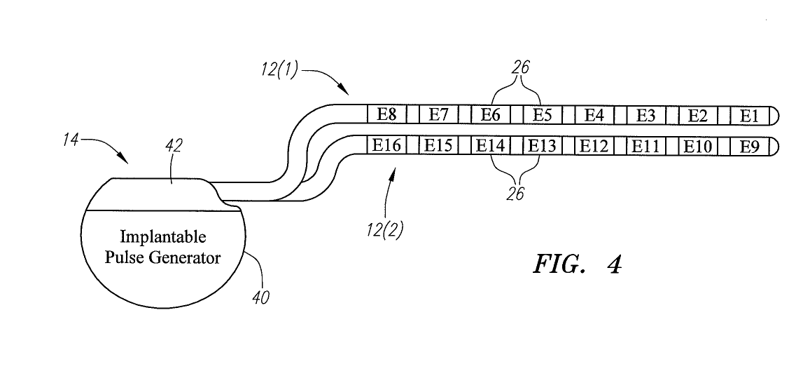

Referring now to FIG. 4, the external features of the stimulation leads 12 and the IPG 14 will be briefly described. One of the stimulation leads 12(1) has eight electrodes 26 (labeled E1-E8), and the other stimulation lead 12(2) has eight electrodes 26 (labeled E9-E16). The actual number and shape of leads and electrodes will, of course, vary according to the intended application. The IPG 14 comprises an outer case 40 for housing the electronic and other components (described in further detail below), and a connector 42 to which the proximal ends of the stimulation leads 12(1) and 12(2) mates in a manner that electrically couples the electrodes 26 to the electronics within the outer case 40. The outer case 40 is composed of an electrically conductive, biocompatible material, such as titanium, and forms a hermetically sealed compartment wherein the internal electronics are protected from the body tissue and fluids. In some cases, the outer case 40 may serve as an electrode.

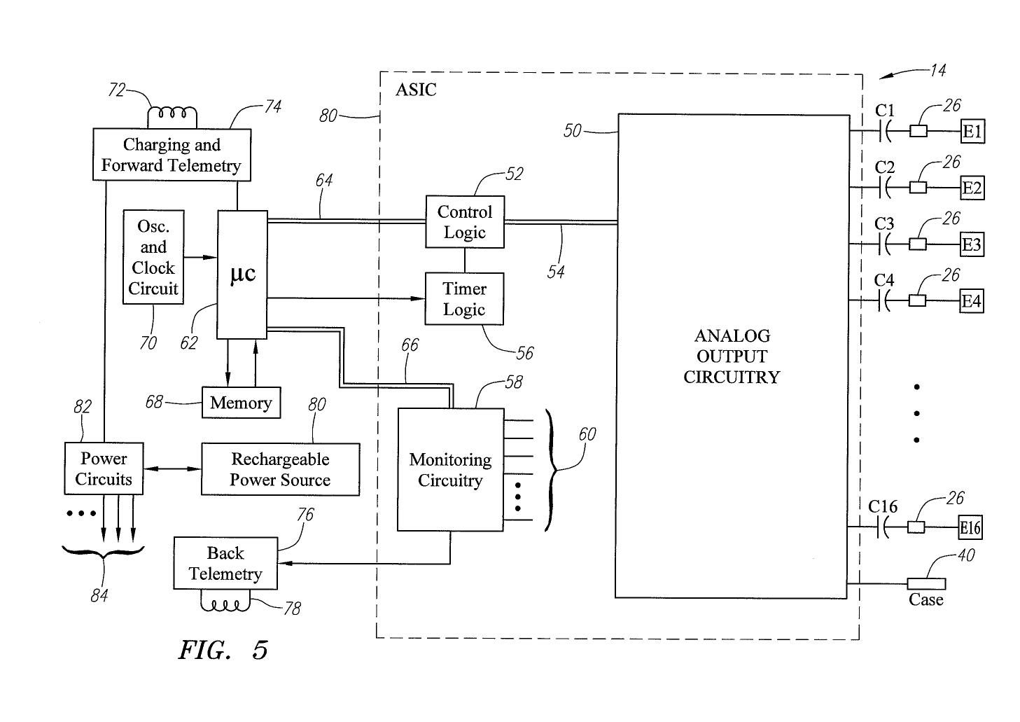

Turning next to FIG. 5, the main internal components of the IPG 14 will now be described. The IPG 14 includes analog output circuitry 50 capable of individually generating electrical stimulation pulses via capacitors C1-C16 at the electrodes 26 (E1-E16) of specified amplitude under control of control logic 52 over data bus 54. The duration of the electrical stimulation (i.e., the width of the stimulation pulses), is controlled by the timer logic circuitry 56. The analog output circuitry 50 may either comprise independently controlled current sources for providing stimulation pulses of a specified and known amperage to or from the electrodes 26, or independently controlled voltage sources for providing stimulation pulses of a specified and known voltage at the electrodes 26. The operation of this analog output circuitry 50, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference.

The IPG 14 further comprises monitoring circuitry 58 for monitoring the status of various nodes or other points 60 throughout the IPG 14, e.g., power supply voltages, temperature, battery voltage, and the like. The monitoring circuitry 58 is also configured for measuring electrical parameter data from which the impedance between the respective electrodes 26 and the IPG case 40 can be computed. Notably, the electrodes 26 fit snugly within the epidural space of the spinal column, and because the tissue is conductive, there is an impedance associated therewith that indicates how easily current flows therethrough. Because implanted electrical stimulation systems depend upon the stability of the devices to be able to convey electrical stimulation pulses of known energy to the target tissue to be excited, measuring electrode impedance is important in order to determine the coupling efficiency between the respective electrode 26 and the tissue.

For example, if the electrode impedance is too high, the respective electrode 26 may be inefficiently coupled to the tissue that it is to stimulate. As a result, an excessive amount of stimulation energy may need to be supplied to the electrode 26 if the analog output circuitry 50 uses current-controlled sources, thereby resulting in an inefficient use of the battery power, or the stimulation energy supplied to the electrode 26 may be otherwise inadequate if the analog output circuitry 50 uses voltage-controlled sources. Other electrical parameter data, such as field potential and evoked action potential, may also be measured to determine the coupling efficiency between the electrodes 26 and the tissue.

Further details discussing the measurement of electrical parameter data, such as electrode impedance, field potential, and evoked action potentials, as well as other parameter data, such as pressure, translucence, reflectance and pH (which can alternatively be used), to determine the coupling efficiency between an electrode and tissue are set forth in U.S. patent application Ser. No. 10/364,436, entitled “Neural Stimulation System Providing Auto Adjustment of Stimulus Output as a Function of Sensed Impedance,” and U.S. patent application Ser. No. 10/364,434, entitled “Neural Stimulation System Providing Auto Adjustment of Stimulus Output as a Function of Sensed Pressure Changes,” which are expressly incorporated herein by reference.

Measurement of the electrode impedance also facilitates fault detection with respect to the connection between the electrodes 26 and the analog output circuitry 50 of the IPG 14. For example, if the impedance is too high, that suggests the connector 42 and/or leads 12 may be open or broken. If the impedance is too low, that suggests that there may be a short circuit somewhere in the connector 42 and/or leads 12. In either event (too high or too low impedance), the IPG 14 may be unable to perform its intended function. Measurement of the electrical parameter data, such as electrode impedance and field potential, also facilitates lead migration detection, as described in U.S. Pat. No. 6,993,384, which has previously been incorporated herein by reference.

Electrical parameter data can be measured using any one of a variety means. For example, the electrical parameter data measurements can be made on a sampled basis during a portion of the time while the electrical stimulus pulse is being applied to the tissue, or immediately subsequent to stimulation, as described in U.S. patent application Ser. No. 10/364,436, which has previously been incorporated herein by reference. Alternatively, the electrical parameter data measurements can be made independently of the electrical stimulation pulses, such as described in U.S. Pat. Nos. 6,516,227 and 6,993,384, which are expressly incorporated herein by reference.

The impedance measurement technique may be performed by measuring impedance vectors, which can be defined as impedance values measured between selected pairs of electrodes 26. The interelectrode impedance may be determined in various ways. For example, a known current (in the case where the analog output circuitry 50 is sourcing current) can be applied between a pair of electrodes 26, a voltage between the electrodes 26 can be measured, and an impedance between the electrodes 26 can be calculated as a ratio of the measured voltage to known current. Or a known voltage (in the case where the analog output circuitry 50 is sourcing voltage) can be applied between a pair of electrodes 26, a current between the electrodes 26 can be measured, and an impedance between the electrodes 26 can be calculated as a ratio of the known voltage to measured current.

The field potential technique may be performed by generating an electrical field at selected ones of the electrodes 26 and recording the electrical field at other selected ones of the lead electrodes 26. This may be accomplished in one of a variety of manners. For example, an electrical field may be generated conveying electrical energy to a selected one of the electrodes 26 and returning the electrical energy at the IPG case 40. Alternatively, multipolar configurations (e.g., bipolar or tripolar) may be created between the lead electrodes 26. Or, an electrode that is sutured (or otherwise permanently or temporarily attached (e.g., an adhesive or gel-based electrode) anywhere on the patient's body may be used in place of the case IPG outer case 40 or lead electrodes 26. In either case, while a selected one of the electrodes 26 is activated to generate the electrical field, a selected one of the electrodes 26 (different from the activated electrode) is operated to record the voltage potential of the electrical field.

The IPG 14 further comprises processing circuitry in the form of a microcontroller (μC) 62 that controls the control logic over data bus 64, and obtains status data from the monitoring circuitry 58 via data bus 66. The IPG 14 additionally controls the timer logic 56. The IPG 14 further comprises memory 68 and oscillator and clock circuitry 70 coupled to the μC 62. The μC 62, in combination with the memory 68 and oscillator and clock circuit 70, thus comprise a microprocessor system that carries out a program function in accordance with a suitable program stored in the memory 68. Alternatively, for some applications, the function provided by the microprocessor system may be carried out by a suitable state machine.

Thus, the μC 62 generates the necessary control and status signals, which allow the μC 62 to control the operation of the IPG 14 in accordance with a selected operating program and stimulation parameters. In controlling the operation of the IPG 14, the μC 62 is able to individually generate stimulus pulses at the electrodes 26 using the analog output circuitry 60, in combination with the control logic 52 and timer logic 56, thereby allowing each electrode 26 to be paired or grouped with other electrodes 26, including the monopolar case electrode, to control the polarity, amplitude, rate, pulse width and channel through which the current stimulus pulses are provided. The μC 62 facilitates the storage of electrical parameter data (or other parameter data) measured by the monitoring circuitry 58 within memory 68, and also provides any computational capability needed to analyze the raw electrical parameter data obtained from the monitoring circuitry 58 and compute numerical values from such raw electrical parameter data for subsequent display to the physician or clinician, as will be described in further detail below.

The IPG 14 further comprises an alternating current (AC) receiving coil 72 for receiving programming data (e.g., the operating program and/or stimulation parameters) from the RC 16 (shown in FIG. 2) in an appropriate modulated carrier signal, and charging and forward telemetry circuitry 74 for demodulating the carrier signal it receives through the AC receiving coil 72 to recover the programming data, which programming data is then stored within the memory 68, or within other memory elements (not shown) distributed throughout the IPG 14.

The IPG 14 further comprises back telemetry circuitry 76 and an alternating current (AC) transmission coil 78 for sending informational data sensed through the monitoring circuitry 58 to the RC 16. The back telemetry features of the IPG 14 also allow its status to be checked. For example, when the RC 16 initiates a programming session with the IPG 14, the capacity of the battery is telemetered, so that the external programmer can calculate the estimated time to recharge. Any changes made to the current stimulus parameters are confirmed through back telemetry, thereby assuring that such changes have been correctly received and implemented within the implant system. Moreover, upon interrogation by the RC 16, all programmable settings stored within the IPG 14 may be uploaded to the RC 16. Significantly, the back telemetry features allow raw or processed electrical parameter data (or other parameter data) previously stored in the memory 68 to be downloaded from the IPG 14 to the RC 16, which information can be used to track the physical activity of the patient.

The IPG 14 further comprises a rechargeable power source 80 and power circuits 82 for providing the operating power to the IPG 14. The rechargeable power source 80 may, e.g., comprise a lithium-ion or lithium-ion polymer battery. The rechargeable battery 80 provides an unregulated voltage to the power circuits 82. The power circuits 82, in turn, generate the various voltages 84, some of which are regulated and some of which are not, as needed by the various circuits located within the IPG 14. The rechargeable power source 80 is recharged using rectified AC power (or DC power converted from AC power through other means, e.g., efficient AC-to-DC converter circuits, also known as “inverter circuits”) received by the AC receiving coil 72. To recharge the power source 80, an external charger (not shown), which generates the AC magnetic field, is placed against, or otherwise adjacent, to the patient's skin over the implanted IPG 14. The AC magnetic field emitted by the external charger induces AC currents in the AC receiving coil 72. The charging and forward telemetry circuitry 74 rectifies the AC current to produce DC current, which is used to charge the power source 80. While the AC receiving coil 72 is described as being used for both wirelessly receiving communications (e.g., programming and control data) and charging energy from the external device, it should be appreciated that the AC receiving coil 72 can be arranged as a dedicated charging coil, while another coil, such as coil 78, can be used for bidirectional telemetry.

As shown in FIG. 5, much of the circuitry included within the IPG 14 may be realized on a single application specific integrated circuit (ASIC) 80. This allows the overall size of the IPG 14 to be quite small, and readily housed within a suitable hermetically-sealed case. Alternatively, most of the circuitry included within the IPG 14 may be located on multiple digital and analog dies, as described in U.S. patent application Ser. No. 11/177,503, filed Jul. 8, 2005, which is incorporated herein by reference in its entirety. For example, a processor chip, such as an application specific integrated circuit (ASIC), can be provided to perform the processing functions with on-board software. An analog IC (AIC) can be provided to perform several tasks necessary for the functionality of the IPG 14, including providing power regulation, stimulus output, impedance measurement and monitoring. A digital IC (DigIC) may be provided to function as the primary interface between the processor IC and analog IC by controlling and changing the stimulus levels and sequences of the current output by the stimulation circuitry in the analog IC when prompted by the processor IC.

It should be noted that the diagram of FIG. 5 is functional only, and is not intended to be limiting. Those of skill in the art, given the descriptions presented herein, should be able to readily fashion numerous types of IPG circuits, or equivalent circuits, that carry out the functions indicated and described, which functions include not only producing a stimulus current or voltage on selected groups of electrodes, but also the ability to measure electrical parameter data at an activated or non-activated electrode.

Additional details concerning the above-described and other IPGs may be found in U.S. Pat. No. 6,516,227, U.S. Patent Publication No. 2003/0139781, and U.S. patent application Ser. No. 11/138,632, entitled “Low Power Loss Current Digital-to-Analog Converter Used in an Implantable Pulse Generator,” which are expressly incorporated herein by reference. It should be noted that rather than an IPG, the SCS system 10 may alternatively utilize an implantable receiver-stimulator (not shown) connected to leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

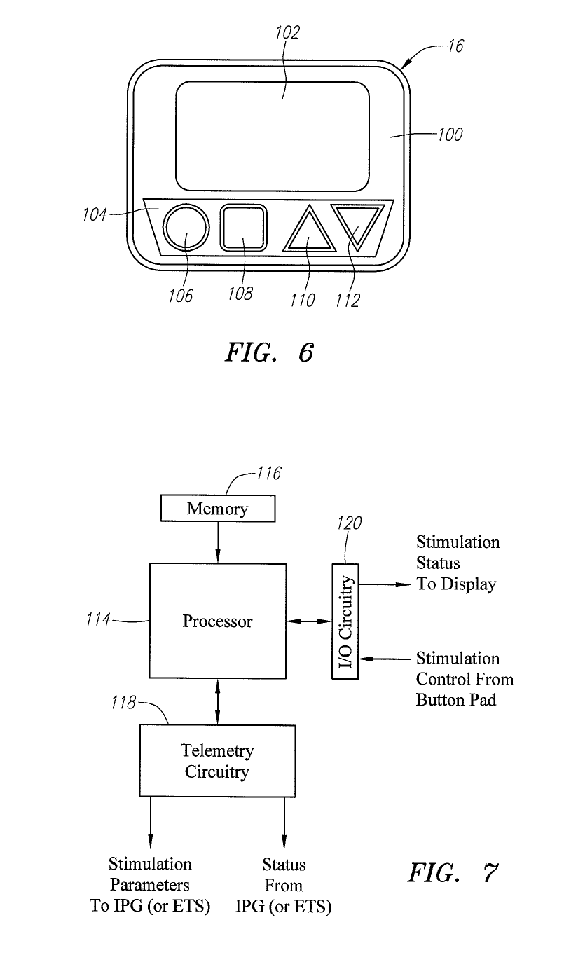

Referring now to FIG. 6, one exemplary embodiment of an RC 16 will now be described. As previously discussed, the RC 16 is capable of communicating with the IPG 14, CP 18, or ETS 20. The RC 16 comprises a casing 100, which houses internal componentry (including a printed circuit board (PCB)), and a lighted display screen 102 and button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. In an optional embodiment, the display screen 102 has touchscreen capabilities. The button pad 104 104 includes a multitude of buttons 106, 108, 110, and 112, which allow the IPG 14 to be turned ON and OFF, provide for the adjustment or setting of stimulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 16 to switch between screen displays and/or parameters. The buttons 110 and 112 serve as up/down buttons that can be actuated to increment or decrement any of stimulation parameters of the pulse generated by the IPG 14, including pulse amplitude, pulse width, and pulse rate. For example, the selection button 108 can be actuated to place the RC 16 in an “Pulse Amplitude Adjustment Mode,” during which the pulse amplitude can be adjusted via the up/down buttons 110, 112, a “Pulse Width Adjustment Mode,” during which the pulse width can be adjusted via the up/down buttons 110, 112, and a “Pulse Rate Adjustment Mode,” during which the pulse rate can be adjusted via the up/down buttons 110, 112. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 7, the internal components of an exemplary RC 16 will now be described. The RC 16 generally includes a processor 114 (e.g., a microcontroller), memory 116 that stores an operating program for execution by the processor 114, as well as stimulation parameter sets in a look-up table, input/output circuitry, and in particular, telemetry circuitry 118 for outputting stimulation parameters to the IPG 14 and receiving status information (including the measured raw or processed electrical parameter data) from the IPG 14, and input/output circuitry, and in particular telemetry circuitry 120, for receiving stimulation control signals from the button pad 104 and transmitting status information to the display screen 102 (shown in FIG. 6). As well as controlling other functions of the RC 16, which will not be described herein for purposes of brevity, the processor 114 generates new stimulation parameter sets in response to the user operation of the button pad 104. These new stimulation parameter sets would then be transmitted to the IPG 14 (or ETS 20) via the telemetry circuitry 118. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the physician or clinician to readily determine the desired stimulation parameters to be programmed into the IPG 14, as well as the RC 16. Thus, modification of the stimulation parameters in the programmable memory of the IPG 14 after implantation is performed by a clinician using the CP 18, which can directly communicate with the IPG 14 or indirectly communicate with the IPG 14 via the RC 16. That is, the CP 18 can be used by the physician or clinician to modify operating parameters of the electrode array 26 near the spinal cord. To facilitate programming of the IPG 14, the CP 18 can be used by the physician or clinician to obtain the measured electrical parameter data from the IPG 14 via the RC 14, which can then be displayed in a manner that can be readily interpreted by the physician or clinician.

As shown in FIG. 3, the overall appearance of the CP 18 is that of a laptop personal computer (PC), and in fact, may be implanted using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

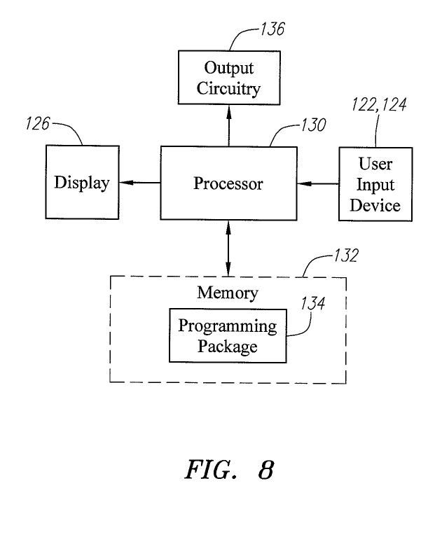

To allow the clinician to perform these functions, the CP 18 includes a mouse 122, a keyboard 124, and a programming display screen 126 housed in a case 128. It is to be understood that in addition to, or in lieu of, the mouse 122, other directional programming devices may be used, such as a joystick, or directional keys included as part of the keys associated with the keyboard 124. As shown in FIG. 8, the CP 18 generally includes a processor 130 (e.g., a central processor unit (CPU)) and memory 132 that stores a stimulation programming package 134, which can be executed by the processor 130 to allow a clinician to program the IPG 14, and RC 16. The CP 18 further includes output circuitry 136 (e.g., via the telemetry circuitry of the RC 16) for downloading stimulation parameters to the IPG 14 and RC 16 and for uploading stimulation parameters, as well as electrical parameter data, already stored in the memory 116 of the RC 16, via the telemetry circuitry 118 of the RC 16. Further details discussing the stimulation programming package 134 are set forth in U.S. patent application Ser. No. ______ (Attorney Docket No. BSC 06-1628-01), entitled “System and Method for Converting Tissue Stimulation Programs in a Format Usable by an Electrical Current Steering Navigator,” which is expressly incorporated herein by reference.

Significantly, the CP 18 acquires the measured electrical parameter data from the IPG 14 via the RC 16, and if not already computed by the IPG 14 or RC 16, computes numerical values from the raw electrical parameter data. The CP 18 then generates a chart representative of the numerical values, and displays this chart to the physician or clinician.

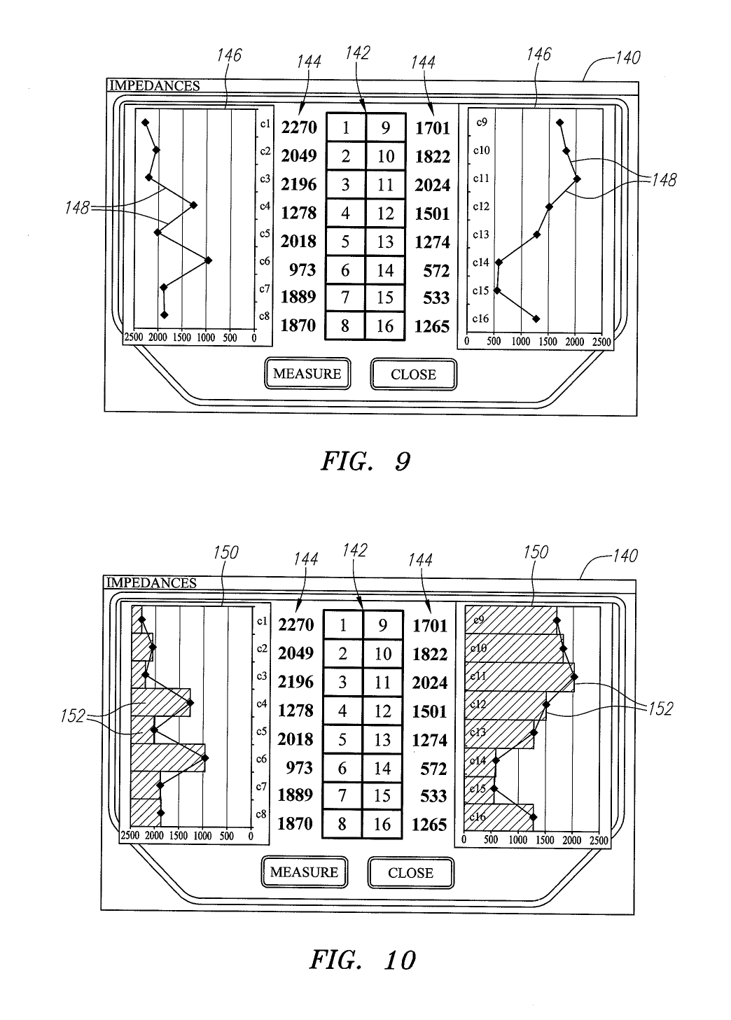

As shown in FIG. 9, an exemplary screen display 140 that can be generated by the CP 18 includes a graphical representation 142 of the electrodes 26 arranged and oriented in the manner in which the electrodes 26 are actually arranged and oriented within the patient. In the graphical electrode representation 142, the electrodes E1-E8 and E9-E16 (i.e., the stimulation leads) are in a side-by-side arrangement, and are oriented from top to bottom in numerical order (i.e., electrodes are numbered from 1 to 8 starting from the top of the first lead, and electrodes are numbered from 9-16 starting from the top of the second lead). As disclosed in U.S. patent application Ser. No. ______ (Attorney Docket No. BSC 06-1628-01), the physician or clinician may, depending on the configuration and orientation of the electrodes 26), select other electrode configurations (e.g., top-bottom configuration) and orientations (e.g., the electrodes may be numbered from 1 to 8 starting from the bottom of the first lead or from 9-16 starting from the bottom of the second lead).

The screen display 140 further comprises a list of the numerical values 144, and in this case impedance values, located adjacent the respective electrodes of the graphical electrode representation 140. Although the list of numerical impedance values 144 provides the clinician or physician an understanding of the coupling efficiency between each of the electrodes 26 and the tissue, to facilitate and expedite such understanding, the screen display 140 further includes a chart 146 representative of the numerical impedance values 144. For purposes of this specification, a chart is defined as a graph or diagram that presents values in non-numerical form. As there shown, the chart 146 is a line chart that plots the magnitudes of the numerical impedance values 144 for the respective electrodes 26, and joins the plotted numerical impedance values 144 with line segments 148. Alternatively, the chart may take the form of another type of chart, such as a bar chart 150 illustrated in FIG. 10. As there shown, the numerical impedance values 144 are represented by bars 152, with the height of the bars 150 defined by the magnitudes of the numerical impedance values 144.

As can be appreciated from FIGS. 9 and 10, the use of charts that plot numerical impedance values provides the physician or clinician a quick view and understanding of the coupling efficiencies between the respective electrodes 26 and the tissue. These coupling efficiencies provide the physician or clinician useful insight into the possible shaping of the electrical stimulation field due to surrounding tissue, and can therefore help to guide programming of the IPG 14 with stimulation parameter sets, as well as to provide insight into the energy usage for the stimulation parameter sets. The physician or clinician may also perform a remedial action with respect to the stimulation provided to the tissue based on the displayed chart; for example, by physically moving one or both of the leads 12 or reprogramming the IPG 14 with new stimulation parameters.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method of providing therapy to a patient implanted with an array of electrodes configured for respectively providing electrical stimulation to tissue of the patient, the method comprising:
measuring physiological parameter information indicative of the coupling efficiencies between the respective electrodes of the array and the tissue;
computing numerical values from the measured physiological parameter information;
generating a chart representative of the computed numerical values; and
displaying the chart to a user.

2. The method of claim 1, wherein the tissue is spinal cord tissue.

3. The method of claim 1, wherein the numerical values are selected from group consisting of electrical impedance values, field potential values, and evoked action potential values.

4. The method of claim 1, wherein the chart is a line chart.

5. The method of claim 1, wherein the chart is a bar chart.

6. The method of claim 1, wherein the physiological parameter information is measured using implanted control circuitry, and the chart is displayed to the user using an external device.

7. The method of claim 6, further comprising transmitting the measured physiological parameter information from the implanted control circuitry to the external device, wherein the numerical values are computed by the external device.

8. The method of claim 6, further comprising programming the implanted control circuitry with a set of stimulation parameters.

9. The method of claim 1, further comprising implanting the array of electrodes within the patient.

10. An external device for a neurostimulation system, comprising:
telemetry circuitry configured for receiving data from an implantable device connected to an array of electrodes, the received data being derived from physiological parameter data measured by the implantable device, the received data being indicative of the coupling efficiencies between respective electrodes of the array and tissue;
processing circuitry configured for generating a chart representative of numerical values derived from the data; and
a display configured for displaying the chart to a user.

11. The external device of claim 10, wherein the numerical values are selected from group consisting of electrical impedance values, field potential values, and evoked action potential values.

12. The external device of claim 10, wherein the received data is the measured physiological parameter data, and the processing circuitry is configured for computing the numerical values from the measured physiological parameter data.

13. The external device of claim 10, wherein the received data comprise the numerical values.

14. The external device of claim 10, wherein the processing circuitry is configured for programming the implantable device with a set of stimulation parameters.

15. The external device of claim 9, wherein the chart is a line chart.

16. The external device of claim 9, wherein the chart is a bar chart.

17. A method of providing therapy to a patient implanted with an array of transducers configured for respectively providing stimulation to tissue of the patient, the method comprising:
measuring physiological parameter information indicative of the efficacy of the stimulation provided to the tissue;
computing numerical values from the measured physiological parameter information;
generating a chart representative of the computed numerical values;
displaying the chart to a user; and
adjusting the stimulation provided by the transducers to the tissue based on the displayed chart.

18. The method of claim 17, wherein the tissue is spinal cord tissue.

19. The method of claim 17, wherein the numerical values are electrical parameter values.

20. The method of claim 17, wherein the chart is a line chart.

21. The method of claim 17, wherein the chart is a bar chart.

22. The method of claim 17, wherein the transducers are electrodes.

23. The method of claim 17, wherein the transducers are initially programmed to adjust the stimulation provided to the tissue.

24. The method of claim 17, wherein a remedial action is performed to adjust the stimulation provided to the tissue.

25. The method of claim 24, wherein the remedial action comprises one or both of physically moving the transducers and reprogramming the transducers.
